(19) United States
(12) Patent Application Publication
(10) Pub. No.: US 2013/0005650 A1
(43) Pub. Date: Jan. 3, 2013

(54) Methods and compositions for treating diseases associated with pathogenic proteins

(76) Inventor: Peter K. Chiang, San Francisco, CA (US)

(21) Appl. No.: 13/540,194

(22) Filed: Jul. 2, 2012

Related U.S. Application Data
(60) Provisional application No. 61/571,597, filed on Jul. 1, 2011.

(51) Int. Cl. A61K 31/473; A61K 31/5513; A61K 38/28; A61P 25/28; A61P 25/16

(52) U.S. Cl. 514/6.5; 514/297; 514/221

(57) ABSTRACT

Methods and compositions are provided comprising acriflavine for inhibiting and treating diseases and disorders associated with pathogenic proteins causing neurodegenerative diseases and amyloid diseases, such as protease resistant prion proteins (PrP^{Sc}) and those associated with transmissible spongiform encephalopathies (TSEs), Alzheimer's disease, amyloidosis, and the like.

### CROSS-REFERENCE TO RELATED APPLICATION

The present application claims priority from U.S. Provisional Patent Application Ser. No. 61/571,597 filed Jul. 1, 2011, which is incorporated by reference herein.

### FIELD

The present disclosure relates to compositions for and methods of treatment of diseases associated with pathogenic proteins, such as, but not limited to: amyloid diseases, such as Alzheimer's disease, protease resistant prion protein (PrP) diseases such as scrapie, transmissible spongiform encephalopathies (TSEs), and disease states where the accumulation, aggregation and/or inherent toxicity of specific proteins are indicated, such as the neurofilaments formed in Amyotropic Lateral Sclerosis, and the like.

### BACKGROUND

Amyloid diseases are diseases or disorders associated with the formation and/or deposition of the pathogenic protein, amyloid. There are many examples of amyloid diseases, and the list includes Alzheimer's disease, systemic senile amyloidosis, prion disease, scrapie, bovine spongiform encephalopathy, Creutzfeldt-Jakob disease, Gerstmann-Straussler-Scheinker syndrome, type I diabetes, adult onset diabetes (type II), insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, lysozyme-associated hereditary systemic amyloidosis, and the like.

Amyloid deposits are found in subjects diagnosed with Alzheimer's disease, a neurodegenerative disease characterized by atrophy of nerve cells in the cerebral cortex, subcortical areas, and hippocampus and the presence of plaques, dystrophic neurites and neurofibrillary tangles. In Alzheimer's disease, dystrophic or aberrant neurite growth, synapse loss, and neurofibrillary tangle formation are strong correlates of disease severity. A typical characteristic of Alzheimer's disease is the presence of amyloid beta (Aβ or Abeta), which is a peptide of 36-43 amino acids that appears to be the main constituent of amyloid plaques (deposits found in the brains of patients with Alzheimer's disease). Similar plaques appear in some variants of Lewy body dementia and in inclusion body myositis (a muscle disease), while Aβ can also form the aggregates that coat cerebral blood vessels in cerebral amyloid angiopathy. The plaques are composed of a tangle of regularly ordered fibrillar aggregates called amyloid fibers, a protein fold shared by other peptides such as the prions associated with protein misfolding diseases. Recent research suggests that soluble oligomeric forms of the peptide may be causative agents in the development of Alzheimer's disease.

Dystrophic neurons characteristically contain abundant electrodense multilaminar bodies in the cytoplasm of the neurites and have disruption of synaptic junctions. The dystrophic neurons surround deposits of amyloid, thereby forming the senile plaques located throughout the brain neuropil as well as in the walls of cerebral blood vessels. An efficacious treatment for Alzheimer's disease is needed that could block or reduce the atrophy of nerve cells, or the formation of senile plaques or neurofibrillary tangles, such that the development of the disease is slowed or arrested.

Amyloid deposits are also found in the islets of Langerhans in patients diagnosed with type II diabetes. The deposits contain an amyloid protein that is derived from a larger precursor called islet amyloid polypeptide (IAPP) or amylin which, in normal animals, has a hormonal role. IAPP is produced by the beta cells of the islets and has a profound effect on glucose uptake by the liver and striated muscle cells. In transgenic mice having a transgene for human amylin and which are fed a high fat diet, overproduction of amylin leads to islet amyloid deposition (see Pathology, 3^{rd} ed. (1999) supra, p. 1226). An efficacious treatment for diabetes type II is needed that could reduce or prevent the formation of amyloid deposits in the islets of Langerhans.

Still another disease where amyloid deposits are noted is amyloid A amyloidosis, in which seemingly unrelated disorders such as chronic inflammatory disorders, neoplastic disorders, and hereditary disorders are linked by a common etiology. The deposition of amyloid protein is secondary to the underlying disease condition. The precursor molecule is serum amyloid A (SAA), an acute phase reactant, which can be used as a surrogate marker of inflammation in many diseases. An efficacious treatment for amyloid A amyloidosis is needed that could reduce or prevent the production of amyloid protein, reduce or prevent the production of the precursor to amyloid protein, prevent or reduce any one of several steps necessary to generate an active amyloid protein, or reduce or prevent the deposition of amyloid plaques, or all of the above.

Yet another disease where amyloid deposits are noted is familial transthyretin amyloidosis, which is the most common form of Familial Amyloidotic Polyneuropathy (FAP). The human amyloid disorders, familial amyloid polyneuropathy, familial amyloid cardiomyopathy and senile systemic amyloidosis, are caused by insoluble transthyretin (TTR) fibrils, which deposit in the peripheral nerves and heart tissue. Transthyretin is a homotetrameric plasma protein implicated in the transport of thyroxine and retinol. The most common amyloidogenic TTR variant is V30M-TTR, while L55P-TTR is the variant associated with the most aggressive form of FAP. An efficacious treatment for amyloidoses caused by transthyretin is needed that could reduce or prevent the production of amyloid protein, reduce or prevent the production of the precursor to amyloid protein, prevent or reduce any one of several steps necessary to generate an active amyloid protein, or reduce or prevent the deposition of amyloid plaques, or all of the above.

A further disease where amyloid deposits are noted is AL amyloidosis, which is a class of diseases related to a primary disorder of immunoglobulin production, including primary amyloidosis, plasma cell dyscrasia, immunoblastic lymphoma, multiple myeloma, and the like. Primary systemic AL (amyloid light-chain) amyloidosis is a plasma cell disorder in which depositions of amyloid light-chain protein cause progressive organ failure. The prognosis of primary amyloidosis is generally poor, with a median survival of 1 to 2 years. The precursor protein in both localized and systemic AL-amyloidosis is an immunoglobulin light chain which shows the same pattern of fragmentation and changes of primary structure. An efficacious treatment for amyloidoses caused by AL amyloid proteins is needed that can reduce or prevent the production of amyloid protein, reduce or prevent the production of the precursor to amyloid protein, prevent or reduce any one of several steps necessary to generate an active amyloid protein, or reduce or prevent the deposition of amyloid plaques, or all of the above.

Yet another disease where amyloid deposits are noted is prion disease, one type of spongiform encephalopathy. Prion diseases are neurodegenerative conditions characterized clinically by progressive ataxia and dementia, and pathologically by vacuolization of spongiform brain tissue. Amyloid deposits are associated with at least one prion disease known as kuru. In kuru, about 70% of prion protein accumulates extracellularly to form plaques, in contrast to normal prion protein which is a constitutively expressed cell-surface glycoprotein (see Pathology, 3^{rd} ed. supra, pp. 1492-1496).

Prion diseases show a common and unique post-translational conversion of a normal-host encoded prion protein (PrP^{C}), to an abnormal disease-causing isoform (PrP^{Sc}), which replicates by stimulating the conversion of PrP^{C} into nascent PrP^{Sc}. The latter is a highly aggregated and detergent insoluble polymer very resistant to proteolysis that can form amyloid. The prion diseases or transmissible spongiform encephalopathies (TSEs) are fatal neurodegenerative diseases in humans and some mammalian species. Non-limiting examples of human prion diseases include Gerstmann-Straussler-Scheinker (GSS) disease, fatal insomnia, and Creutzfeldt-Jakob disease (CJD), and kuru.

The endemics and epidemics of prion diseases in animals in the wild and in the meat industry constitute a serious health problem to the public. Bovine spongiform encephalopathy (BSE) is epidemic in some European countries, and a growing epidemic of chronic wasting disease now afflict deer and elk in North America, while scrapie has been endemic in sheep. The outbreak of a new form of CJD (vCJD) among British youngsters, presumably due to dietary exposure to BSE-contaminated beef, points to a new scenario in the transmission of the deadly prion diseases (Silveira et al. (2004) Curr. Topics. Microbiol. Immunol. 284:1-50).

Treating TSE patients remains a daunting task as there is no therapeutic intervention available and the diseases are uniformly fatal. An efficacious treatment for prion disease is needed that could reduce or prevent the production of amyloid protein, or the deposition of amyloid plaques.

### SUMMARY

Compounds are provided that are useful for preventing, ameliorating, and reversing the effects of diseases and disorders associated with pathogenic proteins, such as amyloid diseases. The compounds include acriflavine, and pharmaceutically acceptable salts, and hydrates thereof.

As disclosed herein, acriflavine has been discovered by Applicant to be an inhibitor of the formation of pathogenic proteins, such as prions, PrP^{Sc} and amyloid fibrils, and as such, can be used to treat pathogenic protein diseases. In some embodiments, methods are provided for reducing and/or clearing pathogenic forms of proteins from cells, by contacting the cells with a therapeutically effective amount of acriflavine.

Disclosed herein are methods of treating a disease characterized by pathogenic protein formation, by administering a pharmaceutically effective amount of acriflavine. Disclosed herein are pharmaceutical compositions comprising acriflavine and a pharmaceutically acceptable carrier or diluent, and optionally at least one additional active agent. An additional pharmaceutically active agent is selected from one or more of a cholinomimetic, a cholinesterase inhibitor, a secretase inhibitor, an NMDA receptor antagonist, an amyloid binding protein, insulin or an insulin mimetic, an insulin sensitizer, a PPAR.gamma. receptor antagonist, colchicine, an anti-inflammatory agent, GSK-3 inhibitor, levodopa, a dopamine receptor agonist, a catechol-O-methyl transferase inhibitor, a MAO-B inhibitor, a muscarinic receptor antagonist, diphenylhydramine HCl, riluzol, baclofen, tizanidine, clonazepam, or dantroline, and the like.

These methods and compositions are useful in the treatment of a disease characterized by pathogenic protein formation such as Creutzfeldt-Jakob disease, scrapie, transmissible spongiform encephalopathy (TSE), Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy, AIDS dementia, systemic senile amyloidosis, prion disease, Gerstmann-Straussler-Scheinker syndrome, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, or lysozyme-associated hereditary systemic amyloidosis.

In additional aspects, methods for reducing or retarding degeneration in a cell population of a tissue or organ subject to damage in an amyloid disease are provided, comprising exposing the cell population to a therapeutically effective amount of acriflavine, and optionally an additional active agent. In an alternative aspect, methods for reducing necrosis or apoptosis in a cell population of a tissue or organ subject to necrotic or apoptotic damage in an amyloid disease are provided, comprising exposing the cell population to a therapeutically effective amount of a compound of acriflavine, and optionally an additional active agent. In particularly preferred embodiments, the cell population comprises pancreatic islet cells, neurons, glia, endothelial cells, or endocrine cells.

In another aspect, methods of blocking amyloid toxicity in cells are provided, comprising contacting said cells with an effective amount of acriflavine, and optionally an additional active agent. Preferably, the amyloid toxicity is selected from amyloid beta peptide toxicity, amyloid prion protein toxicity, human amylin toxicity, amyloid A protein toxicity, transthyretin toxicity, and AL amyloid toxicity.

In some embodiments, treatment is by administering a therapeutically effective amount of the pharmaceutical composition to an animal that has been exposed to and/or is in danger of being exposed to the transmissible agent, such as PrP^{Sc}, or which is exhibiting signs, symptoms or laboratory evidence of a TSE. If the animal is merely suspected of having been exposed to a TSE, the treatment is a prophylactic method of preventing the progression of the disease. In a situation where the animal is already believed to be exhibiting signs or symptoms of the disease, the treatment is also a method of improving the neurological or other biological condition of the animal.

In some aspects of the present disclosure, an animal in need of treatment is identified, and a pharmacologically effective amount of acriflavine is administered to the animal in an amount sufficient to interfere with PrP^{Sc} formation or accumulation in cells.

The present disclosure includes still other aspects comprising treating an animal, including a mammal, such as a human, having a condition associated with PrP^{Sc}. In this aspect, such a mammal is identified and treated with acriflavine, and optionally an additional active agent, in a manner, such as that described herein.

In some aspects of the present disclosure, there is provided a method for reducing or retarding neurodegeneration in a cell population comprising neurons which have been exposed to an amount of a stimulus sufficient to produce partially protease resistant proteins resulting in neurodegeneration; said method comprising administering an efficacious dose of acriflavine, and optionally an additional active agent. In some embodiments, the cell population may reside in the central nervous system of an animal and acriflavine is administered in vivo. The disclosure also provides the use of acriflavine to treat neurodegenerative disease pathology in an animal. In a preferred embodiment, a method is provided for slowing, arresting, or reversing the development of a neurodegenerative disease in a human patient, the method comprising: administering a therapeutically effective amount of acriflavine, and optionally an additional active agent, to the patient to inhibit progression of the disease over a therapeutically effective period of time.

Also provided herein are methods for inhibition of neuronal cell death in a cell population. The methods comprise delivering an effective dosage of acriflavine to a cell population that is exposed to a protease resistant prion protein stimulus.

In an additional aspect, kits are provided for use in treating a disease or disorder associated with pathogenic proteins comprising a container containing a pharmaceutical composition comprising a therapeutically effective amount of acriflavine, and optionally an additional active agent, and instructions comprising guidelines for administration of the composition. Preferably, the instructions comprise an indication of the quantities of the composition to be administered. The kit can further comprise an additional pharmaceutical composition comprising an active agent useful in treating the disease or disorder.

In some aspects of the disclosure, there are provided methods in which acriflavine can be combined with a pharmaceutical product; in a particular a product derived from a human source such as organs, tissue, blood, and related blood derived products. In certain other aspects of the disclosure, there are provided methods of treating tissue, organs, blood and blood derived products by combining such with acriflavine.

In some aspects of the disclosure, there are provided combinations of livestock feed with acriflavine. Non-limiting examples of such feed include meat, bone meal or any material derived from an animal that might be infected with prions.

In certain other aspects of the disclosure, there are provided methods of treating farm animals by administering to farm animals acriflavine combined with livestock feed thereby preventing prion infections and/or treating prion diseases such as “mad cow” disease in animals consuming the treated livestock feed.

### BRIEF DESCRIPTION OF THE FIGURES

FIG. 1 demonstrates the concentration dependent inhibition of Aβ42 fibrillogenesis by acriflavine.

FIG. 2 demonstrates the dose dependent inhibition of PSI⁺ induction by acriflavine.

### DETAILED DESCRIPTION

Definitions and Overview

Before the present compositions and methods are described, it is to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

Unless defined otherwise, all technical and scientific terms used herein have the same meaning as commonly understood by one of ordinary skill in the art to which the methods and compositions as disclosed herein belongs. Although any methods and materials similar or equivalent to those described herein can also be used in the practice or testing of the present methods, the preferred methods and materials are now described.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

It must be noted that as used herein and in the appended claims, the singular forms “a,” “and” and “the” include plural referents unless the context clearly dictates otherwise. Thus, for example, reference to “a compound” includes a plurality of such compounds and reference to “the step” includes reference to one or more steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided might be different from the actual publication dates, which may need to be independently confirmed.

The term “pathogenic” is used in its conventional sense, and refers to the quality of causing disease or of being capable of causing disease (Merriam-Webster's Medical Desk Dictionary, 1996).

The term “pathogenic protein” refers to proteins that are malformed (i.e., insoluble or denatured), misprocessed, toxic, infectious, or that otherwise exhibit a deleterious effect on health and/or biological function. Such proteins may have residual activity or no activity, but in all cases are associated with disease and pathogenesis in the affected animal or human patient.

The pharmaceutically acceptable acid addition salts of compounds of acriflavine may be used in the various compositions and methods described herein. Acriflavine is basic in nature and is thus capable of forming a wide variety of salts with various inorganic and organic acids. The acids that may be used to prepare pharmaceutically acceptable acid addition salts of acriflavine are those that form non-toxic acid addition salts, i.e., salts containing pharmacologically acceptable anions, such as, but not limited to, the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, fumarate, gluconate, glucaronate, saccharate, formate, benzoate, glutamate, methanesulfonate, ethanesulfonate, benzenesulfonate, and p-toluenesulfonate. Acriflavine may be converted into pharmaceutically acceptable salts by methods well known to those skilled in the art.

The term “therapeutically effective amount,” as used herein, refers to an amount sufficient to detectably treat, ameliorate, prevent or retard the progression of an unwanted condition or symptom associated with disorders related to pathogenic proteins. In some instances, the amount is that determined to provide an in vitro effect sufficient to treat, ameliorate, prevent or detectably retard the progression of an unwanted condition or symptom.

The terms “treatment,” “treating,” “treat” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in an animal, particularly a human, and includes: (a) preventing the disease or symptom from occurring in a subject which may be predisposed to the disease or symptom but has not yet been diagnosed as having it; (b) inhibiting the disease of its symptom, i.e., arresting development of the disease or its symptoms; or (c) relieving the disease symptom, i.e., causing regression of the disease or symptom.

Applicant has also surprisingly demonstrated a significant reduction in the yeast prion [PSI⁺] expression, i.e. amyloid formation, by acriflavine in a yeast cell model system. Acriflavine inhibits the amyloid aggregation of yeast prion in a dose-dependent manner in vitro.

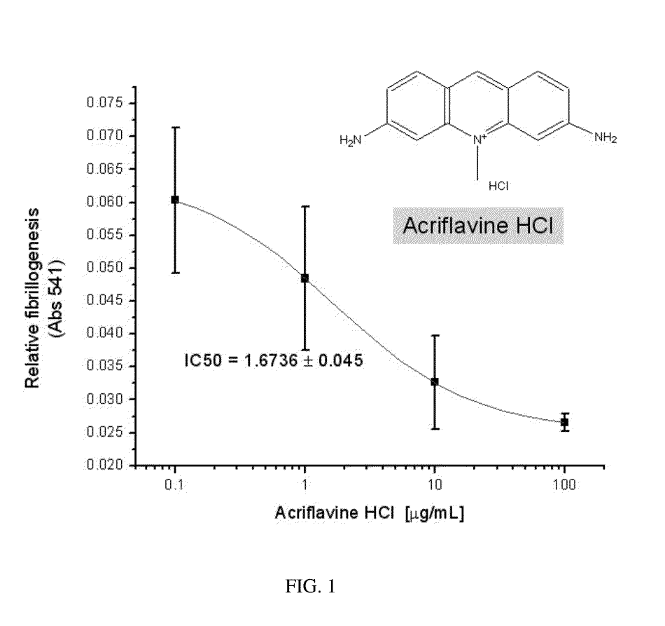

Applicant has further surprisingly discovered that acriflavine is effective to reduce or reverse amyloid fibril formation (Aβ42 fibrillogenesis) in vitro. As shown in the Examples and FIG. 1, acriflavine was effective in reducing the ability of amyloid peptides in solution to form fibrils, and to cause an increase in the amount of amyloid peptide monomer after incubation of acriflavine with amyloid fibrils.

Accordingly, an animal suffering from a condition associated with pathogenic amyloid fibril formation such as, for example, Alzheimer's disease, can be treated by administration of acriflavine, optionally comprising an additional active agent, as disclosed herein.

Compounds

In some embodiments, the present disclosure relates to compositions containing an acridine derivative.

The acridine derivative according to some embodiments includes acriflavine, acriflavine neutral, acridine orange, acridine yellow G, diacridine, aniline mustard, salts thereof, and the like. Acriflavine neutral as one of the acridine derivatives is a compound composed of 3,6diamino-10-methylacridium chloride and 3,6-diaminoacridine in the mixing ratio of approximately 1:1 and is a yellowish-brown powder having the following chemical structure:

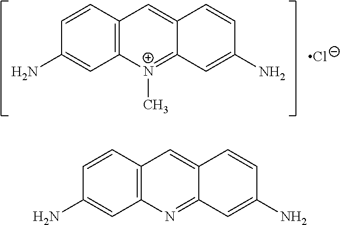

Acriflavine neutral which used as the acridine derivative in some embodiments herein is a dye having an acridine nucleus and has a week basic property, and is fluorescent in aqueous solution. This compound has been used as an anti-malarial, anti-tripanosomal or bactericidal agent and inhibits the growth of microorganisms generally at the concentrations of 10⁻⁴ to 10⁻⁵ M. Since Ehrlich synthesized acriflavine neutral from acridine nucleus in 1912, anti-tripanosomal activity of the agent has been found. Thus, acriflavine neutral has been used externally or systemically for treatment of purulent diseases due to its potent antibacterial activity. Acriflavine neutral has a low toxicity at the clinically used dosage range, although the agent weakly irritates mucous membranes. Thus, it can be used in the form of injectable or enteral medicines. In contrast to other antibacterial agents, acriflavine neutral is characterized by its antibacterial activity which is not lowered in the presence of serum proteins, blood, etc. Furthermore, acriflavine neutral has a rapid onset of action, and does not suppress the hematopoietic system, nor damage the liver, etc., following repetitive administrations for a prolonged period.

The mechanism of action of acriflavine neutral against microorganisms is not firmly established. However, it has been reported that since the structure of acriflavine neutral is similar to that of riboflavine (vitamine B₂), acriflavine neutral inhibits diamino acid oxidase, etc., as the enzyme present in trypanosoma, due to metabolic antagonistic activity, and is ionized in the living body to produce a cationic part which inhibits bacterial respiratory enzymes (see, e.g. Keigiro Takaki, Pharmacology (Japan), p617, 1989).

Acriflavine can be obtained commercially from a variety of sources (e.g., MP Biomedical, LLC under catalog numbers: 02190675, 02190675, 05211381 and 05211372)

Compositions

Compositions provided herein include pharmaceutically acceptable dosage forms for oral, parenteral, inhalation, transdermal and topical administration, as discussed further below. Such dosage forms can optionally comprise pharmaceutically acceptable carriers, diluents, and the like, and such formulations and formulation methods are well known to those of skill in the art, e.g., in solution, tablet, suppository, injectable, and nasal spray.

Compositions and kits can also include an additional active agent useful for the treatment of diseases and conditions associated with pathogenic proteins described herein. For treatment, amelioration and/or prevention of Alzheimer's disease, compositions can comprise a pharmaceutically acceptable dosage form comprising acriflavine alone or in combination with an additional active agent useful for preventing, ameliorating or reversing Alzheimer's disease, or for treating the symptoms associated with Alzheimer's disease. Additional active agents include but are not limited to those selected from a secretase inhibitor, such as α- or β-secretase inhibitor, including but not limited to compounds described in U.S. Pat. No. 6,586,475 to Kato, U.S. Pat. No. 7,119,085 to Hom, or a γ-secretase inhibitor; an acetylcholinesterase inhibitor, including but not limited to donepezil (ARICEPT); amyloid binding proteins, such as an antibody having specific binding for amyloid; neuroprotective agents such as the moderate affinity NMDA receptor antagonists, including by not limited to memantine; and other agents typically used in the treatment of patients with Alzheimer's disease such as psychotropics, estrogen, statins, nonsteroidal anti-inflammatory agents, and antioxidants.

For treatment, amelioration and/or prevention of diabetes type I, compositions can comprise a pharmaceutically acceptable dosage form comprising acriflavine and an additional active agent useful for preventing, ameliorating or reversing diabetes, or alone or in combination with treating the symptoms associated with diabetes. Additional active agents include but are not limited to a glycogen synthase kinase 3 (GSK-3) inhibitor, such as a thiadiazolidinone (e.g., NP031112 (NeuroPharma), insulin or insulin mimetic, insulin sensitizer, such as metformin or the PPAR-γ receptor antagonists, such as the thiazolidinediones (e.g., rosiglitazone (AVANDIA) or pioglitazone (ACTOS, Takeda), or anti-hyperglycemic agents such as glucophage (METFORMIN) or sulfonylureas such as glipizide (GLUCOTROL).

For treatment, amelioration and/or prevention of diabetes type II, compositions can comprise a pharmaceutically acceptable dosage form comprising acriflavine alone or in combination with an additional active agent useful for preventing, ameliorating or reversing diabetes, or for treating the symptoms associated with diabetes. Additional active agents include but are not limited to a glycogen synthase kinase 3 (GSK-3) inhibitor, such as a thiadiazolidinone (e.g., NP031112 (NeuroPharma), insulin or insulin mimetic, insulin sensitizer, such as metformin or the PPAR-γ receptor antagonists, such as the thiazolidinediones (e.g., rosiglitazone (AVANDIA) or pioglitazone (ACTOS, Takeda), or anti-hyperglycemic agents such as glucophage (METFORMIN) or sulfonylureas such as glipizide (GLUCOTROL); and insulin or an insulin mimetic.

For treatment, amelioration and/or prevention of Parkinson's disease, compositions can comprise a pharmaceutically acceptable dosage form comprising acriflavine alone or in combination with an additional active agent useful for preventing, ameliorating or reversing Parkinson's disease, or for treating the symptoms associated with Parkinson's disease. Additional active agents include but are not limited to levodopa, dopamine receptor agonists, such as bromocriptine; catechol-O-methyl transferase (COMT) inhibitors such as tolcapon; MAO-B inhibitors, such as selegelline; muscarinic receptor antagonists such as trihexyphenidyl, diphenylhydramine HCl; or Amantadine.

For treatment, amelioration and/or prevention of TSE, compositions can comprise a pharmaceutically acceptable dosage form comprising acriflavine alone or in combination with an additional active agent useful for preventing, ameliorating or reversing TSE, or for treating the symptoms associated with TSE.

For treatment, amelioration and/or prevention of CJD, compositions can comprise a pharmaceutically acceptable dosage form comprising acriflavine alone or in combination with an additional active agent useful for preventing, ameliorating or reversing CJD, or for treating the symptoms associated with CJD.

For treatment, amelioration and/or prevention of amyotrophic lateral sclerosis (ALS), compositions can comprise a pharmaceutically acceptable dosage form comprising acriflavine alone or in combination with an additional active agent useful for preventing, ameliorating or reversing ALS, or for treating the symptoms associated with ALS. Additional active agents include but are not limited to riluzol, baclofen, tizanidine, clonazepam, and dantroline.

For treatment, amelioration and/or prevention of multiple sclerosis, compositions can comprise a pharmaceutically acceptable dosage form comprising acriflavine alone or in combination with an additional active agent useful for preventing, ameliorating or reversing MS, or for treating the symptoms associated with MS.

For treatment, amelioration and/or prevention of Huntington's disease, compositions can comprise a pharmaceutically acceptable dosage form comprising acriflavine alone or in combination with an additional active agent useful for preventing, ameliorating or reversing Huntington's disease, or for treating the symptoms associated with Huntington's disease.

For treatment, amelioration and/or prevention of systemic amyloidosis, compositions can comprise a pharmaceutically acceptable dosage form comprising acriflavine and an additional active agent useful for preventing, ameliorating or reversing systemic amyloidosis, or for treating the symptoms associated with systemic amyloidosis. Additional active agents include but are not limited to colchicine.

For treatment, amelioration and/or prevention of primary amyloidosis, compositions can comprise a pharmaceutically acceptable dosage form comprising acriflavine alone or in combination with an additional active agent useful for preventing, ameliorating or reversing systemic amyloidosis, or for treating the symptoms associated with systemic amyloidosis. Additional active agents include but are not limited to colchicine.

For treatment, amelioration and/or prevention of diffuse Lewy body disease, compositions can comprise a pharmaceutically acceptable dosage form comprising acriflavine alone or in combination with an additional active agent useful for preventing, ameliorating or reversing Lewy body disease, or for treating the symptoms associated with Lewy body disease.

For treatment, amelioration and/or prevention of Pick's disease, compositions can comprise a pharmaceutically acceptable dosage form comprising acriflavine alone or in combination with an additional active agent useful for preventing, ameliorating or reversing Pick's disease, or for treating the symptoms associated with Pick's disease.

For treatment, amelioration and/or prevention of fronto-temporal dementia, compositions can comprise a pharmaceutically acceptable dosage form comprising acriflavine alone or in combination with an additional active agent useful for preventing, ameliorating or reversing fronto-temporal dementia, or for treating the symptoms associated with fronto-temporal dementia.

For treatment, amelioration and/or prevention of AIDS dementia, compositions can comprise a pharmaceutically acceptable dosage form comprising acriflavine alone or in combination with an additional active agent useful for preventing, ameliorating or reversing AIDS dementia, or for treating the symptoms associated with AIDS dementia.

Additional indications could include, but are not limited to, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, lysozyme-associated hereditary systemic amyloidosis, autism, schizophrenia, bipolar disorders, progressive supranuclear palsy, systemic lupus erythematosus, rheumatoid arthritis, spinocerebellar ataxias, Crohn's disease, ulcerative colitis, and polyneuropathy. Compositions can comprise a pharmaceutically acceptable dosage form comprising acriflavine alone or in combination with an additional active agent useful for preventing, ameliorating or reversing each of these conditions.

Methods of Treatment

The methods and compositions disclosed herein are useful in the treatment of an animal suffering from a disease or condition resulting from or associated with a pathogenic protein. The animal is not particularly limited, and can be a farm animal, for example, a cow, pig, sheep, chicken or goat, and the like, a companion animal, for example, a dog, cat, parrot, and the like, a zoo animal, for example, an elephant, or tiger, and the like, a laboratory animal, for example, a rat, mouse, transgenic mouse, and the like, a wild animal for example, a bird, fish, reptile, and the like), or a game animal, for example, elk, deer, pheasant, and the like. In some embodiments, the animal is a mammal, including a human patient.

In some embodiments, pharmaceutical compositions comprising acriflavine, with or without additional active agent, can be administered to a patient over a significant period of time when the patient is infected with a prion related disease such as CJD. This treatment is a non-prophylactic treatment, but rather a direct treatment of the patient, such as a human, in an attempt to cure what is now a 100% fatal disease. In some embodiments, there are provided methods for reducing or retarding degeneration in a cell population of a tissue or organ subject to damage in an amyloid disease, comprising exposing the cell population to a therapeutically effective amount of acriflavine. In some embodiments, there is provided a method for reducing necrosis or apoptosis in a cell population of a tissue or organ subject to necrotic or apoptotic damage in an amyloid disease, comprising exposing the cell population to a therapeutically effective amount of acriflavine. The cell population can comprise any cell exhibiting damage, necrosis or apoptosis associated with an amyloid disease, and typically includes pancreatic islet cells, neurons, glia, endothelial cells, or endocrine cells.

In some embodiments, there are provided methods of blocking amyloid toxicity in cells, said methods comprising contacting said cells with an amount of acriflavine effective to inhibit formation of amyloid oligomers and polymers. Preferably, the amyloid toxicity is selected from amyloid beta peptide toxicity, amyloid prion protein toxicity, human amylin toxicity, amyloid A protein toxicity, transthyretin toxicity, or AL amyloid toxicity.

In some embodiments, methods are provided for reducing or retarding neurodegeneration in a cell population comprising neurons which have been exposed to an amount of a stimulus sufficient to produce at least partially protease resistant proteins resulting in neurodegeneration, the methods comprising: exposing the cell population to a therapeutically effective amount of acriflavine. Similarly, methods are provided for inhibition of neuronal cell death in a cell population that is exposed to a protease resistant prion protein stimulus, the method comprising: administering a therapeutically effective amount of acriflavine to the cell population.

Diseases and Associated Pathogenic Proteins

Any disease that is etiologically linked to the formation and/or deposition of amyloid is contemplated for treatment according to the present invention. Amyloid diseases or disorders are associated with the formation and/or deposition of amyloid, and include, but are not limited to, Creutzfeldt-Jakob disease, scrapie, transmissible spongiform encephalopathy (TSE), Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy, AIDS dementia, systemic senile amyloidosis, prion disease, Gerstmann-Straussler-Scheinker syndrome, insulinoma, amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, or lysozyme-associated hereditary systemic amyloidosis, and the like. Acriflavine can be used in a method of treatment of these amyloid diseases by administering a therapeutically effective amount of acriflavine and optionally a pharmaceutically acceptable carrier or diluent, and/or an additional active agent.

Methods of treating pathogenic protein diseases, like protease resistant prion related diseases, are also provided. Diseases that may be treated by the claimed methods include, but are not limited to TSE, CJD, Kuru, Alzheimer's disease, Parkinson's disease, amyotrophic lateral sclerosis, multiple sclerosis, autism, schizophrenia, bipolar disorders, fronto-temporal dementia, Pick's disease, progressive supranuclear palsy, diffuse Lewy body disease, systemic lupus erythematosus, rheumatoid arthritis, Huntington's disease, spinocerebellar ataxias, diabetes mellitus, Types I and II, Crohn's disease, ulcerative colitis, systemic amyloidosis, primary amyloidosis, polyneuropathy and AIDS dementia.

Certain examples provided herein relate to the use of acriflavine in the reduction and/or clearance of PrP^{Sc} from cells. Accordingly, in certain embodiments, the compositions and methods as described herein can be applied to obtain the reduction and/or clearance of the formation of pathogenic proteins for any protein which assumes two different conformational shapes, one of which is associated with the disease. The following is a non-limiting list of diseases with associated insoluble proteins which assume two or more different conformations:

| Disease | Pathogenic Proteins |
|---|---|
| Alzheimer's Disease | APP, Aβ peptide, α1- |
| | antichymotrypsin, tau, non-Aβ |
| | component |
| Prion diseases, Creutzfeld Jakob | Prions (PrP^{x}) including but |
| disease, scrapie and Bovine | not limited to hPrP and |
| Spongiform Encephalopathy | PrP^{Sc} |
| ALS | SOD and neurofilament |
| Pick's disease | Pick body |
| Parkinson's disease | Lewy body |
| Diabetes Type 1 | Amylin |
| Multiple myeloma-plasma cell | IgGL-chain |
| dyscrasias | |
| Familial amyloidotic polyneuropathy | Transthyretin |
| Medullary carcinoma of thyroid | Procalcitonin |
| Chronic renal failure | β₂-microglobulin |
| Congestive heart failure | Atrial natriuretic factor |
| Senile cardiac and systemic myloidosis | Transthyretin |
| Chronic inflammation | Serum amyloid A |
| Atherosclerosis | ApoA1 |
| Familial amyloidosis | Gelsolin |
| Huntington's Disease | Huntington protein (mHtt) |

Thus, a “pathogenic protein” is selected from amyloid proteins such as, but not limited to, APP, Aβ peptide, α1-antichymotryPSIn, tau, non-Aβ component, PrP^{Sc}, SOD, neurofilament, Pick body, Lewy body, amylin, IgGL-chain, transthyretin, procalcitonin, β₂ 2-microglobulin, atrial natriuretic factor, serum amyloid A, apoA1, gelsolin or Huntington protein.

It should be noted that the proteins listed above each include a number of variants or mutations which result in different strains which are all encompassed by the present disclosure. Known pathogenic mutations and polymorphisms in the PrP gene related to prion diseases are given below and the sequences of human, sheep and bovine are given in U.S. Pat. No. 5,565,186, issued Oct. 15, 1996. The following is a list of such mutations and polymorphisms:

| Pathogenic human | Human | Sheep | Bovine |
|---|---|---|---|
| mutations | Polymorphisms | Polymorphisms | Polymorphisms |
| 2 octarepeat insert | Codon 129 | Codon 171 | 5 or 6 |
| | Met/Val | Arg/Glu | octarepeats |
| 4 octarepeat insert | Codon 219 | Codon 136 | |
| | Glc/Lys | Ala/Val | |
| 5 octarepeat insert | | | |
| 6 octarepeat insert | | | |
| 7 octarepeat insert | | | |
| 8 octarepeat insert | | | |
| 9 octarepeat insert | | | |
| Codon 102 Pro-Leu | | | |
| Codon 105 Pro-Leu | | | |
| Codon 117 Ale-Val | | | |
| Codon 145 Stop | | | |
| Codon 178 Asp-Asn | | | |
| Codon 180 Val-Ile | | | |
| Codon 198 Phe-Ser | | | |
| Codon 200 Glu-Lys | | | |
| Codon 210 Val-Ile | | | |
| Codon 217 Asn-Arg | | | |
| Codon 232 Met-Ala | | | |

It should also be noted that such proteins have two different 3-dimensional conformations with the same amino acid sequence. One conformation is associated with disease characteristics and is generally insoluble whereas the other conformation is not associated with disease characteristics and is soluble. The methodology described herein is not limited to the diseases, proteins and strains listed.

Prophylactic Uses

As indicated herein, the term “treating” means preventing, inhibiting or relieving the disease or symptom thereof. However, there are specific situations wherein a clear prophylactic treatment is indicated, i.e., the aspect of treating that involves preventing the disease. Specifically, there is an identifiable portion of the population that has inherited diseases related to prions such as CJD. Family members who have or might have an inherited trait for such disease can take a composition as described herein in order to prevent the development of the disease and/or symptoms. A second class of individuals are those who have been exposed to prions by ingesting infected food such as beef products, which were derived from cattle with BSE, and includes individuals who have spent significant amounts of time in Europe and/or other areas where the cattle were likely to have been infected with BSE. A third group of people are people who have been treated with human growth hormone derived from a human cadaver. Such individuals are at risk for the development of such diseases and would be treated with a composition as described herein. A fourth group of individuals are those that have been subjected to surgery and exposed their nerve tissue to surgical instruments, which may have been already infected with prions. Further, the individual may be an individual who has had a dura mater transplant from an individual who may have been infected with a disease such as CJD.

In treating patients who do not at the present have an amyloid disease such as a prion disease or Alzheimer's disease, but who are believed to be at substantial risk for developing such diseases, the physician should start treatment when the patient first experiences early symptoms, such as memory or cognitive problems, in Alzheimer's disease, or high blood sugar in diabetes type II, for example. In addition, there are some patients who may be diagnosed with an amyloid disease through the detection of a biological or genetic marker predictive for the amyloid disease. For example, patients having the genetic marker for APOE4 are at risk for developing Alzheimer's disease. In these situations, even though the patient does not have symptoms of the disease, the administration of pharmaceutical compositions comprising acriflavine, with or without additional active agent, may be started before symptoms appear and treatment can be continued indefinitely to prevent or delay the outset of the disease.

Modes of Administration and Dosages

The compositions as described herein may be administered to the patient by a variety of different means. The means of administration will vary depending upon the intended application. As one skilled in the art would recognize, administration of the compositions can be carried out in various fashions, for example, via topical administration, including, but not limited to, dermal, ocular and rectal; transdermal, via passive or active means, e.g., using a patch, a carrier, or iontophoresis; transmucosal, e.g., sublingual, buccal, rectal, vaginal, or transurethral; oral, e.g., gastric or duodenal; parenteral injection into body cavity or vessel, e.g., intraperitoneal, intravenous, intralymphatic, intratumoral, intramuscular, interstitial, intraarterial, subcutaneous, intralesional, intraocular, intrasynovial, intraarticular; or via inhalation, e.g., pulmonary or nasal inhalation, using e.g., a nebulizer. Oral administration is preferred in certain patient classes, while for others, rectal or parenteral administration may be preferred. Oral administration can utilize immediate release or controlled release compositions. The mode of administration will generally affect dosing in that some modes are more efficient at delivery of active compound to the desired site than others.

Direct infusion methods, and use of osmotic pumps, which administer a composition as disclosed herein directly to cells to be treated (e.g., brain tissue), can also be used.

Any suitable dosage can be given in the methods described herein. Acriflavine and the carrier and the amount will vary widely depending on the species of the animal, body weight, and disease being treated. The dosage administered will, of course, vary depending upon known factors, such as the pharmacodynamic characteristics of acriflavine and the mode and route of administration; the age, health and/or weight of the recipient; the nature and extent of the symptoms; the kind of concurrent treatment; the frequency of treatment; and the effect desired, for example. Those skilled in the art will adjust dosing as needed, beginning with smaller doses and increasing gradually while monitoring side effects and the effect of the drug on the disease being treated. With an oral composition of acriflavine, dosing can be generally in an amount of about 0.5 mg to about 10,000 mg/day/75 kg of body weight of the animal being treated. With an oral composition of acriflavine HCl, for example, a human dose can be about 0.1-1,000 mg/day, and larger animals are given larger doses in proportion to their weight. Typically, the therapeutic amount is in the range of from about 0.1 to about 1000 mg/day, or in the range of from about 15 to about 1500 mg/day, or in the range of from about 1 to about 100 mg/day, in the range of from about 5 to about 50 mg/day, or in the range of 0.5 to 2 mg/day.

Dosages of agents co-administered with acriflavine are selected based on usual dosages known for these agents and adjusted as necessary based on individual patient health and needs, drug-drug interactions, and other contraindications.

Pharmaceutical Compositions and Kits

The present disclosure further concerns pharmaceutical compositions incorporating acriflavine. Such pharmaceutical compositions should contain a therapeutic or prophylactic amount of acriflavine. The pharmaceutically acceptable carrier can be any compatible, non-toxic substance suitable to deliver the compounds to an intended host. Pharmaceutically acceptable adjuvants, buffering agents, dispersing agents, and the like may also be incorporated into the pharmaceutical compositions. Preparation of pharmaceutical conditions is well described in the medical and scientific literature. See, for example, Remington's Pharmaceutical Sciences, Mack Publishing Company, Easton, Pa., 16^{th} Ed., 1982.

Acriflavine may be administered alone or in combination with pharmaceutically acceptable carriers or diluents by any of the routes previously indicated, and such administration may be carried out in single or multiple doses. More particularly, the therapeutic compounds used in the methods of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, pessary, jellies, gels, pastes, lotions, ointments, elixirs, syrups, and the like. Such carriers include solid diluents or fillers, sterile aqueous media and various non-toxic organic solvents, for example.

Capsules or tablets can be easily formulated and can be made easy to swallow or chew; other solid forms include granules, and bulk powders. Tablets can contain suitable binders, lubricants, diluents, disintegrating agents, coloring gents, flavoring agents, flow-inducing agents, and melting agents. For oral administration, tablets containing various excipients such as, but not limited to, microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For oral administration in liquid dosage form, acriflavine can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Oral dosage forms optionally contain sweeteners, flavorants and coloring agents. Parenteral and intravenous forms can also include minerals and other materials to make them compatible with the type of injection or delivery system chosen. Thus, the present disclosure provides compositions for administration to a host, where the compositions comprise a pharmaceutically acceptable solution of acriflavine in an acceptable carrier, as described above.

Gelatin capsules can contain acriflavine and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Lipophilic solvents or carriers, such as DMZ, an organic solvent, and lipids such as phosphatidyl choline or cholesterol can be utilized. Solutions for parenteral administration preferably contain a water soluble salt of acriflavine or a liposome, suitable stabilizing agents, and if necessary, buffer substances. Antioxidizing agents such as sodium bisulfate, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules

For parenteral administration, solutions of a therapeutic compound used in the methods of the present invention can be prepared in substances selected from, but not limited to, oils, such as sesame or peanut oil, or aqueous propylene glycol, or any other pharmaceutically acceptable formulation may be employed. The aqueous solutions should be suitably buffered if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

Acriflavine can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Acriflavine can also be coupled with soluble polymers as targetable drug carriers. Such polymers can include polyvinylpyrrolidone, pyran copolymer, polyhydroxylpropylmethacrylamidephenol, polyhydroxyethylaspartamidephenol, or polyethyleneoxide-polylysine substituted with palmitoyl residues. Furthermore, acriflavine can be coupled to a class of biodegradable polymers useful in achieving controlled release of a drug, for example, polylactic acid, polyglycolic acid, copolymers of polylactic and polyglycolic acid, polyePSIIon caprolactone, polyhydroxy butyric acid, polyorthoesters, polyacetals, polydihydropyrans, polycyanoacylates, and crosslinked or amphipathic block copolymers of hydrogels.

The pharmaceutical compositions just described are suitable for systemic administration to the host, including parenteral, topical, and oral administration, as well as intracranial administration.

A unit dosage can comprise acriflavine in a mixture with other compounds or other inhibiting compounds. The dosage unit can also comprise diluents, extenders, carriers and the like. The unit can be in solid or gel form such as pills, tablets, capsules and the like or in liquid form suitable for oral, rectal, topical, intravenous injection or parenteral administration or injection.

Acriflavine can be mixed with a pharmaceutically acceptable carrier. This carrier can be a solid or liquid and the type is generally chosen based on the type of administration being used. Acriflavine can be co-administered with additional pharmaceutically active agents in the form of a tablet or capsule, as an agglomerated powder or in a liquid form. Examples of suitable solid carriers include lactose, sucrose, gelatin and agar.

Compositions containing acriflavine can be administered for prophylactic and/or therapeutic treatments of neurodegenerative disease. In therapeutic application, compositions are administered to a patient already affected by the particular neurodegenerative disease in an amount sufficient to cure or at least partially arrest the condition and its complications. An amount adequate to accomplish this is defined as a “therapeutically effective dose” or “efficacious dose.”

Acriflavine can be an effective pharmaceutical for therapy in pathogenic protein diseases, like prion related diseases in mammals. Acriflavine is known to cross the blood-brain barrier and PrP^{Sc} inhibiting concentrations of acriflavine can be achieved in the brain by daily dosages.

In methods as disclosed herein, acriflavine can form the active ingredient, and is typically administered in admixture with suitable pharmaceutical diluents, excipients, or carriers (collectively referred to herein as a pharmaceutically acceptable carrier or carrier materials) suitably selected with respect to the intended form of administration, that is, oral tablets, capsules, elixirs, syrups and the like, and consistent with conventional pharmaceutical practices. For instance, for oral administration in the dosage unit form of a tablet or capsule, acriflavine can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like.

Acriflavine is able to cross the blood-brain barrier of a human to produce a therapeutically efficacious concentration in cerebrospinal fluid and CNS tissues (e.g., cortical neurons). Other approaches to enhancing delivery of compounds of acriflavine, particularly across the blood-brain barrier, can utilize osmotic pumps and/or pharmacologic-based procedures involving drug latentiation or the conversion of hydrophilic drugs into lipid-soluble drug.

In some embodiments, a physiologically compatible salt of acriflavine can be used in the methods described herein. Non-limiting examples of such salts include organic and inorganic acids, as exemplified by hydrochloric acid, sulfuric acid, phosphoric acid, formic acid, acetic acid, oxalic acid, tartaric acid, maleic acid, benzoic acid, succinic acid, fumaric acid, levulinic acid, salicyclic acid, citric acid, isocitric acid, adipic acid, lactic acid, α-ketoglutaric acid, malic acid, malonic acid, glyceric acid, mevalonic acid, glucuronic acid, neuraminic acid, glutaric acid, aspartic acid, gluconic acid, mandelic acid, ascorbic acid, lactobionic acid, glucoheptonic acid, glutamic acid, nicotinic acid, pantothenic acid, folic acid, adenylic acid, geranylic acid, cytidylic acid and inosic acid.

The present disclosure also provide pharmaceutical kits useful, for example, for the treatment of pathogenic protein diseases, which comprise one or more containers containing a pharmaceutical composition comprising a therapeutically effective amount of acriflavine, with or without additional active agents. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit. In the present disclosure it should be understood that the specified materials and conditions are important in practicing the methods disclosed herein but that unspecified materials and conditions are not excluded so long as they do not prevent the benefits of the disclosed methods from being realized.

The present disclosure also provides the use of acriflavine to slow, arrest, or reverse the development of a degenerative diseases, including a neurodegenerative disease, in a human patient; an efficacious amount of acriflavine with or without additional active agent is administered to the patient to inhibit progression of the disease.

Livestock Feed

Some aspects of the present disclosure concern combinations of acriflavine combined with a livestock feed. Acriflavine can be combined with a livestock feed derived from an animal source, such as meat or bone meal and more particularly animal material that includes ground material from the central nervous system from another animal. Such feed may be infected with prions. However, acriflavine can be combined with plant derived materials used in livestock feed in order to prevent or treat prion infections in the animal eating the feed. More particularly, acriflavine can be added to animal feed or feed stuffs used to feed any type of animal and particularly for livestock. The feed stuff compositions disclosed herein are intended to provide nutritional requirements of a variety of animals, including cattle, poultry, swine, sheep, goats, other monogastric or ruminant livestock. The compositions generally vary according to the type of animals to which the feedstuff will be given. Examples of various animal feedstuff components can be found in U.S. Pat. No. 6,207,217, U.S. Pat. No. 6,203,843, U.S. Pat. No. 5,786,007, U.S. Pat. No. 4,225,621, U.S. Pat. No. 4,161,543 and U.S. Pat. No. 4,062,988.

Generally, when the term “feedstuff” is used with respect to the present disclosure, the term comprises all types of plant and animal components. In some embodiments, “feedstuff” includes organic components such as proteins, crude fiber, acid detergent fiber, neutral detergent fiber, vitamins and minerals. Typical compositions of feedstuffs for livestock include, but are not limited to, the following components: alfalfas, ammonium sulfate, barleys, beet pulps, blood meal, bluestem grass, brewers grains and yeast, brome grass, calcium carbonate, canary grass, carrot pulp, roots and tops, cattle manure, cheatgrass, clovers, coffee grounds, corn and corn plants, cottonseed, defluorinated phosphate, diammonium phosphate, dicalcium phosphate, distillers grain barley, distillers grain corn, feathermeal hydrolyzed, garbage (municipal), grain screenings and grain dust, grape pomace, grass silage, hominy feed, hop leaves, vines and spent hops, limestone, linseed meal, all types of hay including meadow hay, meat and bone meal (MBM), milo grain, mint slug silage, molasses beet, cane, citrus and wood, monoammonium phosphate, mono-dicalcium phosphate, navy beans, all types of oats including oat hay, oat silage, oat straw, oat grain, groats, oat meal, oat mill byproducts and oat hull, orange pulp, orchard grass, pea vines, peanut hulls, skins and meal, potato vine, potatoes and potato waste, poultry fat and poultry litter and manure, prairie hay, rapemeal solvent, rye straw and grain, safflower meal, sagebrush, sorghum stover and silage, soybeans and soybean hull, sudangrass hay and silage, sunflower meal and hulls, timothy hay and silage, tomatoes, triticale silage, urea, wheat bran, wheat grass, wheat grain, wheat shorts and wheat straw. Further, the above feedstuff components are set forth above serve merely as examples and are not intended to be comprehensive or limiting. As such, suitable feedstuffs for the present disclosure may comprise additional components not provided in the list above.

Of the above listed feed components, meat and bone meal (MBM) stands out as one the richest sources of energy and minerals. Typically, the crude protein content of MBM is about 50%. See, Hamilton, C. R., “Meat and Bone Meal,” Esteem Products. Vol. 1 (1). MBM is thus one of the most efficient feed components. MBM is produced as a by-product from the removal of fat from animal tissues through rendering. The rendering process produces a finely ground, dry residue of animal by-products pressure cooked and stabilized by high temperature steam in closed tanks. The fat can be skimmed off and the solid residue is pressed to remove as much of the fat and water as possible. As defined and regulated by the Association of American Feed Control Officials (AAFCO), MBM is the rendered product from mammal tissues, including bone, exclusive of any added blood, hair, hoof, horn, hide trimmings, manure, stomach and rumen contents, except in such amounts as may occur unavoidably in good processing practices. As such, neuronal tissues are included in MBM products. See, “The BSE Inquiry” §9.15 at www.bse.org.uk/report/volume7/chapteh2.htm.

The present disclosure encompasses feedstuff as defined, in combination with acriflavine. In some embodiments, acriflavine is added to feedstuff and fed to an animal and in particular to domesticated livestock farm animals such as cows, pigs, sheep, goats, horses, chickens, etc. Acriflavine can be added in an amount sufficient to “treat” the animal. The amount will vary based on factors such as the type of animal and its size. In some embodiments, dosing is such that the animal will receive about 0.1 mg to about 10,000 mg/day/kg of weight of the animal.

Methods of Determining Efficacy of Acriflavine Against Amyloid Formation

One skilled in the art is able to practice the screening procedures required to determine whether or not acriflavine or related compound possesses activity against pathogenic proteins.

Yeast prion (PSI⁺) transmission is mediated by the self propagating aggregation of the translation termination factor Sup35, which is comprised of a prion-determining region (NM) fused to a translational termination region, that results in a nonsense suppression phenotype (Uptain, et al. (2001) EMBO J. 20:6236-6245). Polymerization of Sup35 is mediated by the yeast Sup-NM protein, which consists of glutamine/asparagine-rich N-terminal domain (N) and a charged middle domain (M) (Bradley et al. (2004) Mol. Microbiol. 51:1649-1659, forming self replicating amyloid fibers (Glover et al. (1997) Cell 89:811-819; King et al. (1997) Proc. Natl. Acad. Sci. U.S.A. 94:6618-6622). When introduced into yeast these fibers initiate the (PSI⁺) prion state (Sparrer et al. (2000) Science 289, 595-599; King et al. (2004) Nature 428, 319-323; Tanaka et al. (2004) Nature 428:323-328), showing that the amyloids are in fact the infectious yeast prion element underlying (PSI⁺). Sup-NM amyloid formation in vitro reflects closely the induction and propagation of Sup35 in vivo, and PSI⁺ variants resemble the mammalian prion strains in that they can propagate in the same host strain with different disease latencies and pathological manifestations (Uptain et al., 2001).

As the yeast prion state (PSI⁺) is an excellent model for delineating amyloid fiber formation and prion propagation (Collins et al. (2004) PLoS Biol. 2, e321), polymerization assays of the yeast prion Sup-NM can be performed to demonstrate activity of agents against yeast prion formation and propagation. Prions are self-propagating in nature and amyloid formation is common in most, but not all TSEs. Therefore, the yeast prion model is likely applicable to prion formation and propagation in other systems, including TSEs affecting mammals and birds.

In addition, the inhibition of the oligomerization of amyloid peptide (inhibition of amyloid fibrillogenesis) can be estimated by the method described by Klunk, et al. using Congo Red (Klunk, W. E., Jacob, R. F., Mason, R. P. (1999) “Quantifying amyloid β-peptide aggregation using the congo red-Aβ spectrophotometric assay” Anal. Biochem. 266:66-76). This assay method was used to demonstrate inhibition of fibrillogenesis by acriflavine. Inhibition of amyloid fibrillogenesis and disassociation of fibrils can also be demonstrated using a gel assay.

It is to be understood that while the invention has been described in conjunction with some embodiments thereof, that the description above as well as the examples that follow are intended to illustrate and not limit the scope of the invention. The practice of the present invention will employ, unless otherwise indicated, conventional techniques of organic chemistry, polymer chemistry, biochemistry and the like, which are within the skill of the art. Other aspects, advantages and modifications within the scope of the invention will be apparent to those skilled in the art to which the invention pertains. Such techniques are explained fully in the literature.

### EXAMPLES

The following examples are put forth so as to provide those of ordinary skill in the art with a complete disclosure and description of how to make and use the presently disclosed compositions and methods, and are not intended to limit the scope of what the Applicant regard as the invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All solvents were purchased as HPLC grade, and all reactions were routinely conducted under an inert atmosphere of argon unless otherwise indicated. Unless otherwise indicated, the reagents used were obtained from the following sources: amyloid peptide gels, and gel stains from Invitrogen (Camarillo, Calif.), organic solvents, from Aldrich Chemical Co., Milwaukee, Wis.; gases, from Matheson, Seacaucus, N.J.

### Example 1

Acriflavine Exhibits an Inhibitory Effect on Amyloid Fibrillogenesis

The inhibition of the oligomerization of amyloid peptide was estimated by the method described by Klunk, et al. using Congo Red (Klunk, W. E., Jacob, R. F., Mason, R. P. (1999) “Quantifying amyloid β-peptide aggregation using the congo red-Aβ spectrophotometric assay” Anal. Biochem. 266, 66-76). Briefly, amyloid peptides were prepared in 40 μM in Tris-Acetate (40 mM:8 mM) buffer. Acriflavine HCl to be tested for inhibition of fibrillogenesis was dissolved in phosphate buffered saline, and then diluted 100 fold into Tris-Acetate buffer. Aliquots of 100 μl of amyloid peptide were incubated with acriflavine at room temperature for 5 days before measuring the absorbance. BSA was included in the buffer as a negative control. After incubation, 20 μl of 35 μM congo red was added to the 100 μl of incubated amyloid peptides to a final concentration of 7 μM. The absorbance of Congo Red at 541 nm was read using a Perkin-Elmer Victor3 1420 Multilabel Counter. The inhibition of oligomerization was calculated using the following formula: Inhibition=(Abs₅₄₁ of sample-Abs₅₄₁ of control)/(Abs₅₄₁ of Amyloid peptide-Abs₅₄₁ of BSA).

As shown in FIG. 1, acriflavine HCl exhibited a concentration-dependent inhibition of amyloid fibrillogenesis. The IC₅₀ for inhibition of fibrillogenesis was determined to be approximately 1.7 μg/mL.

### Example 2

Acriflavine Inhibits Yeast Amyloid Polymerization

Polymerization assays of the yeast prion protein Sup-NM with or without acriflavine were performed using thioflavin T measurements as described by Collins, et al., (PLoS Biol. 2(10):e321). Concentrated stocks of NM protein stored in either 6 M GdnHCl or 4 M GdnHCl and 4 M urea were diluted at least 200-fold into buffer C (5 mM potassium phosphate and 150 mM sodium chloride [pH 7.4]). Sup-NM seed was added immediately before observation. Fluorescence was monitored in a fluorescence plate reader (Molecular Devices, Sunnyvale, Calif., USA; 442 nm excitation and 483 nm emission). Reactions were carried out at 25° C. with 3 seconds of shaking between each measurement.

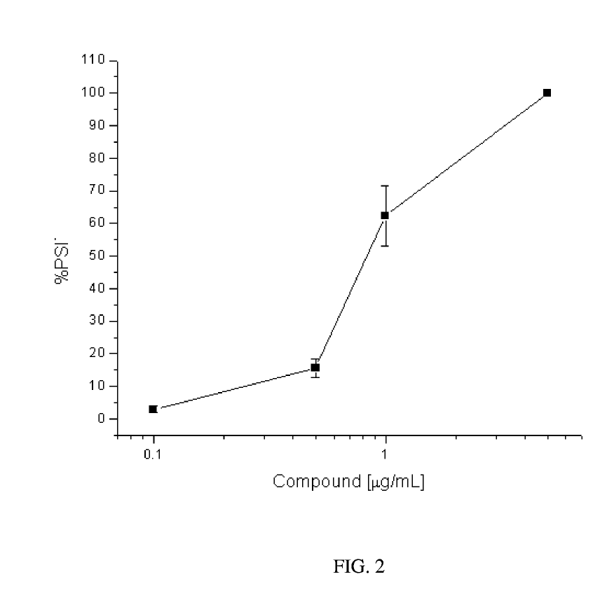

Acriflavine was able to inhibit in a dose-dependent manner the amyloid formation of yeast prion protein, Sup-NM. FIG. 2 demonstrates the inhibition curve as a function of acriflavine concentration expressed as % control of the induction of PSI⁺ in yeast.

PSI⁺ Induction Assay in Yeast

The induction of yeast PSI⁺ (the yeast prion protein Sup35) was performed as described in published methods (Santoso et al. (2000) Cell 100:277-288). Briefly, PST cells containing a pRS426 Cp-NM-GFP plasmid were incubated for 24 h with 50 μM copper sulfate plus or minus drugs at 30° C. with shaking. After incubation each cell mixture was diluted 5000-fold in water, from which 100 μL was added to two different types of agar plates: (1) SD Complete (both PSI⁺ and PST cells were able to grow on these plates; (2) SD-Ade (only PSI⁺ cells were able to grow on the plates lacking adenine (Ade)). The plates were incubated at 30° C. for 48 h or until the yeast colonies became visible. The number of colonies was next counted on all plates to determine the fraction of cells that have converted to the PSI⁺ prion state (obtained by dividing the number of colonies on the SD-Ade plate by the number of colonies on the SD Complete plate).

The results are shown in FIG. 2, which demonstrates the concentration dependent inhibition of PSI⁺ induction in yeast cells by acriflavine.

### Example 3

Acriflavine neutral 60 mg

In a light resistant container, 60 mg of acriflavine neutral was dissolved in 40 ml of warm distilled water, cooled, adjusted to pH 6.5-7.0 and then sterilized. The resulting sterilized solution is used as an injectable preparation. The injectable preparation is warmed approximately to body temperature before use.

Unless defined otherwise, all technical and scientific terms used herein have the same meaning as commonly understood by one of ordinary skill in the art to which this disclosure belongs. Unless mentioned otherwise the techniques employed or contemplated herein are standard methodologies well known to one of ordinary skill in the art. The materials, methods and examples are illustrative only and not limiting. All numerical ranges in this specification are intended to be inclusive of their upper and lower limits.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

What is claimed is:

1. A method of treating an animal suffering from a disease or condition associated with pathogenic proteins, comprising administering to said mammal a pharmaceutical composition comprising a therapeutically effective amount of acriflavine and pharmaceutically acceptable salts and hydrates thereof.

2. The method of claim 1, wherein the animal is a mammal, including a human patient.

3. The method of claim 2, wherein the human patient is suffering from Alzheimer's disease or Parkinson's disease.

4. The method of claim 2, wherein the human patient is suffering from Alzheimer's disease.

5. The method of claim 1, wherein said acriflavine comprises acriflavine HCl.

6. The method of claim 1, wherein said acriflavine comprises acriflavine neutral.

7. The method of claim 1, wherein said pharmaceutical composition further comprises at least one additional pharmaceutically active agent.

8. The method of claim 7, wherein the additional pharmaceutically active agent is selected from one or more of a cholinomimetic, a cholinesterase inhibitor, a secretase inhibitor, an NMDA receptor antagonist, an amyloid binding protein, insulin or an insulin mimetic, an insulin sensitizer, a PPARγ receptor antagonist, colchicine, an anti-inflammatory agent, GSK-3 inhibitor, levodopa, dopamine receptor agonist, catechol-O-methyl transferase inhibitor, MAO-B inhibitor, muscarinic receptor antagonist, diphenylhydramine HCl, riluzol, baclofen, tizanidine, clonazepam, or dantroline.

9. A method for slowing, arresting, or reversing the development of Alzheimer's disease in a human patient, the method comprising: administering a therapeutically effective amount of acriflavine to the patient to inhibit progression of the disease over a therapeutically effective period of time.

10. A kit for use in treating disease or disorder associated with Alzheimer's disease, the kit comprising: a container containing a pharmaceutical composition comprising a therapeutically effective amount of acriflavine, and instructions comprising guidelines for administration of the composition according the method of claim 1.

11. A pharmaceutical composition for treating a mammal suffering from Alzheimer's disease, comprising a therapeutically effective amount of acriflavine; and pharmaceutically acceptable salts and hydrates thereof; and optionally a pharmaceutically acceptable carrier.
